(19) United States
(12) Patent Application Publication
(10) Pub. No.: US 2006/0289017 A1
(43) Pub. Date: Dec. 28, 2006

(54) Adjustable restraints

(76) Inventors: Ryan D. Schmeltz, South Bend, IN (US); Daniel P. Collins, Mishawaka, IN (US)

Correspondence Address: BAKER & DANIELS LLP, 205 W. JEFFERSON BOULEVARD, SUITE 250, SOUTH BEND, IN, 46601, US

(21) Appl. No.: 11/452,142

(22) Filed: Jun. 13, 2006

Related U.S. Application Data
(60) Provisional application No. 60/689,880, filed on Jun. 13, 2005.
(60) Provisional application No. 60/689,880, filed on Jun. 13, 2005.

(51) Int. Cl. A61B 19/00

(52) U.S. Cl. 128/869

(57) ABSTRACT

An adjustable restraining device configured for use to restrain an individual. The restraining device allows for the distance separating restraints to be adjusted. The restraining device may include a pair of clamps separated by a maximum distance. The clamps may be attached to the wrists of the individual and a first set distance representing an allowable distance separating the clamps of the device may be adjusted, as desired. The device also includes a buckle assembly that allows for the shortening of the first set distance, when desired, but only allows the increasing of the first set distance upon actuation of the buckle.

### This application claims priority from U.S. Provisional Application Ser. No. 60/689,880, filed Jun. 13, 2005, which is incorporated herein by reference.

### BACKGROUND OF THE INVENTION

1. Field of the Invention

This invention relates generally to handcuffs, ankle cuffs, or similar type restraints. More specifically, the invention relates to devices that allow a user to adjust the distance separating handcuffs, ankle cuffs, or similar type restraints.

2. Description of the Related Art

U.S. Pat. No. 1,534,936 teaches a Confining and Restraining Device. The disclosed device includes two metal parts or rings, a female member and a male member. A belt, cord or the like connects the female member to the male member. The purpose of the device is to easily confine the arms of a person against his body. The first ring of the device is adjustably connected to the male member, and the ring is adjustably connected to the female member. The rings are also fixed to the ends of the belt. A person, when secured by the device, is entrapped by the belt and the combination of the male member and the female member. The belt or cord may be tightened around the person by pulling on the rings.

U.S. Pat. No. 5,345,947 discloses a Wrist and Ankle Secured Restraining Device. The restraining device is configured for use in securing a handcuffed prisoner. The restraining device includes a control strap extending through a releasable cam buckle. The cam buckle is resiliently secured to handcuffs. A selected amount of the control strap is looped around the ankles of a prisoner and extends through the cam buckle in order to expose a control end. The control end represents the end of the strap that may be pulled by a restrainer when the restrainer desires to shorten the distance separating the ankles and handcuffs of the prisoner. The restrainer may actuate the cam buckle in order to release the control strap and increase the distance separating the ankles and handcuffs of the prisoner.

U.S. Pat. No. 5,551,447 discloses a Restraint Belt. The restraint belt disclosed secures the arms and legs of a detainee while also providing the detainee a limited amount of movement under the complete control of a detainer. The device includes an elongated belt having a belt buckle connection to secure the belt to the waist of the detainee. In addition, the device includes arm restraints, each including a tether and a handcuff. Each arm restraint is independently attached to the belt by way of a tether buckle. The device may also optionally include an adjustable leg tether with buckles for restraining one or both legs. The leg buckles and handcuffs all have locks that are preferably operable by a single key. The locks control the releasability of each restraint or tether. The tethers allow the detainee to have limited freedom for walking, fingerprinting, holding objects, etc., while remaining under the control of the detainer. The length of the belt is adjustable.

U.S. Pat. No. 5,651,376 discloses a Flexible Dual Loop Restraining Device. The adjustable restraining device includes first and second flexible straps, each of which is formed into a loop. The first and second straps are connected in a slideable manner to each other. Each strap includes a head portion and an opposing end portion. The head portion of each strap includes first and second slots. The loops are formed in each strap by inserting the end portion of the strap through one of the slots in the head portion. The straps are interconnected with each other by inserting the free end of each strap through the second of the slots. When the restraining device is used to restrain a prisoner, the loops of the device are drawn closer to one another by grasping and pulling the free ends of the straps.

U.S. Pat. No. 6,026,661 discloses a Restraining Device and Method of Using the same. The restraining device disclosed includes a pair of restraints, preferably handcuffs. Elongated cables connect the handcuffs separately to a housing assembly. The cables are also attached to a rotatable spool housed within a box ratchet assembly. The method of using the restraint includes separately applying each restraint to the wrists of a arrestee. The cables may then be reeled into the spool member with a ratcheting motion. This method of utilizing the device enables a law enforcement official to bring the wrists of the arrestee together in a controlled fashion.

### SUMMARY OF THE INVENTION

Embodiments of the present invention are a restraining device including a first clamp connected to a strap and a second clamp connected to a buckle assembly configured to receive the strap. In embodiments of the invention, the buckle assembly can freely traverse the strap in a first direction.

In embodiments of the invention, the buckle assembly may traverse the strap in a second direction opposite the first direction when actuated. In addition, the buckle assembly may be actuated by a key. Furthermore, the clamps may be adjusted in size when the clamps are actuated by the key.

In embodiments of the invention, the buckle assembly includes a latch and a cover. The cover may include a pair of slots. In embodiments of the invention, a first of the slots is formed in a surface including a keyhole and a second of the slots is formed in a surface opposite the surface including the keyhole. In embodiments of the invention, the pair of slots are formed in a single surface.

In embodiments of the invention, the restraining device further includes a third clamp connected to a first clamp and a fourth clamp connected to the second clamp. The restraining device may also include a first chain connecting the first clamp to the third clamp and a second chain connecting the second clamp to the fourth clamp. In embodiments of the invention, the restraining device may further include a pair of rings. The first ring may connect the first chain to the strap, and the second ring may connect the second chain to the buckle assembly.

In embodiments of the invention, the strap may include a loop. The loop may be formed in the strap opposite the first clamp.

In embodiments of the invention, the restraining device includes a first clamp connected to a strap and a second clamp connected to a buckle assembly. The strap may include a loop. In embodiments of the invention, a set distance separates the first clamp and the second clamp. The set distance may decrease when the loop is pulled.

In embodiments of the invention, the set distance increases when the buckle assembly is actuated.

In embodiments of the invention, the buckle assembly may include at least one slot and the strap may extend through the at least one slot.

In embodiments of the invention, the buckle assembly may include a latch and a locking pawl. The latch prevents movement of the strap through the buckle assembly. The locking pawl prevents the movement of the latch.

In embodiments of the invention, the buckle assembly further includes a strap receiving area. A portion of the strap may be located intermediate the strap receiving area and the latch.

### BRIEF DESCRIPTION OF THE DRAWINGS

FIG. 1 is a perspective view of an adjustable restraint accordingly to one embodiment of the invention.

FIG. 2 is a perspective view of a clamp utilized in the embodiment of the invention depicted in FIG. 1.

FIG. 3 is a perspective view of a buckle assembly utilized in embodiments of the invention.

FIG. 3A is an underside perspective view of the buckle assembly depicted in FIG. 3.

FIG. 4 is an exploded perspective view of the buckle assembly shown in FIG. 3.

FIGS. 5 is a section view of the buckle assembly depicted in FIG. 4.

FIGS. 6A-6C depict views of the embodiment of FIG. 1 in use restraining an individual.

FIG. 7 is a section view of the buckle assembly depicted in FIG. 5 being actuated with a key.

FIG. 8 is an exploded perspective view of an adjustable restraint according to another embodiment of the invention.

FIG. 9 is a perspective view of the embodiment of FIG. 8 when in use in restraining an individual.

FIG. 10 is a perspective view of the individual depicted in FIG. 9 in a restrained position.

FIGS. 11-13 depict section views of an alternative embodiment of the buckle assembly utilized in embodiments of the present invention.

FIG. 14 is an exploded perspective view of an alternative embodiment of a cover that may be used with embodiments of a buckle assembly.

### DETAILED DESCRIPTION OF THE EMBODIMENTS OF THE INVENTION

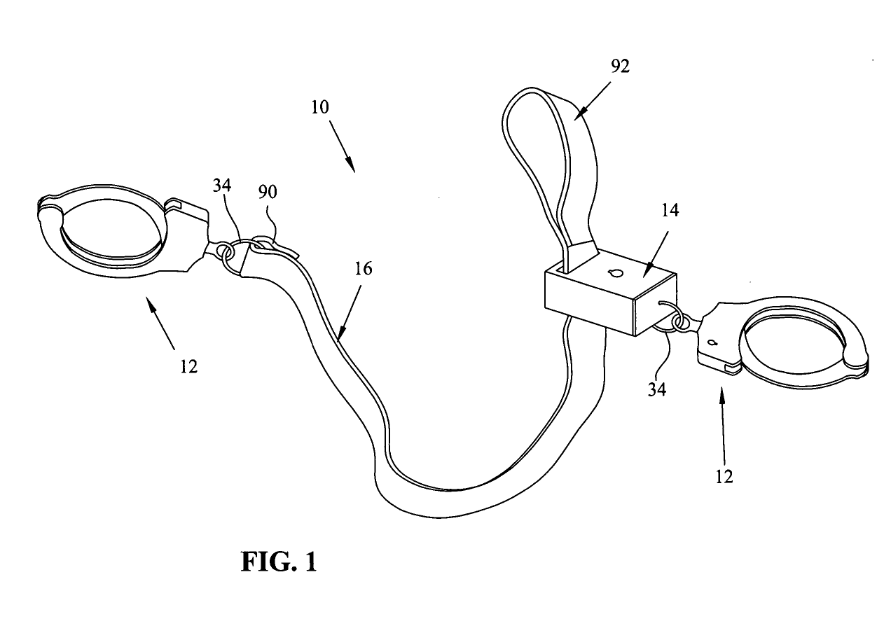

FIG. 1 depicts a perspective view of an adjustable restraint, generally indicated by numeral 10. The adjustable restraint 10 includes a pair of clamps, each generally indicated by numeral 12, a buckle assembly 14 and a strap 16.

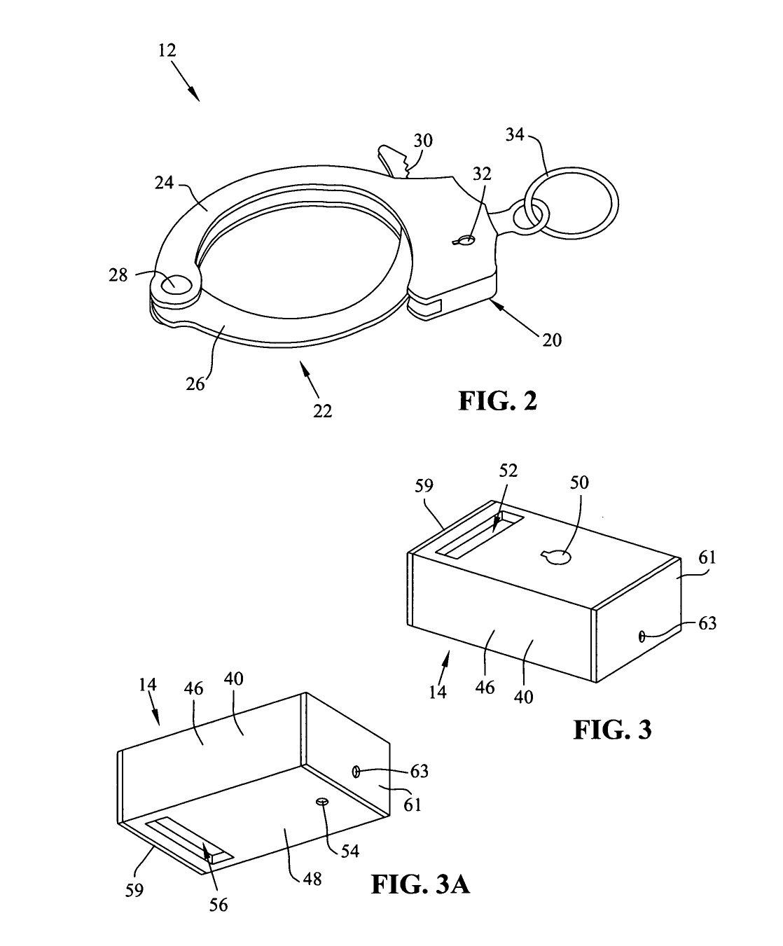

FIG. 2 depicts a perspective view of a clamp 12. Clamp 12 may be any type of clamp known in the art. In the present embodiment, clamp 12 includes a base 20 and a receiving portion 22. Receiving portion 22 comprises a fixed arm 24 extending outward from base 20 and a pivot arm 26. Pivot 28 connects fixed arm 24 to pivot arm 26. In the embodiment of the invention depicted, pivot arm 26 includes a plurality of teeth generally indicated by numeral 30.

In the present embodiment of the invention, base 20 includes a key hole 32 and a ring 34. Key hole 34 is sized and configured to receive a key (not shown). Ring 34 is positioned within base 20 and located opposite the receiving portion 22. Key hole 32 allows a user to access a latching mechanism (not shown) with the key. The latching mechanism mates with teeth 30 of pivot arm 26 in a known manner. Specifically, the latching mechanism allows the pivot arm 26 to travel through base 20 in only one direction. Once the key is inserted into key hole 32 and rotated in a known manner, the latching mechanism allows the pivot arm 26 to freely travel within the latching mechanism in any direction.

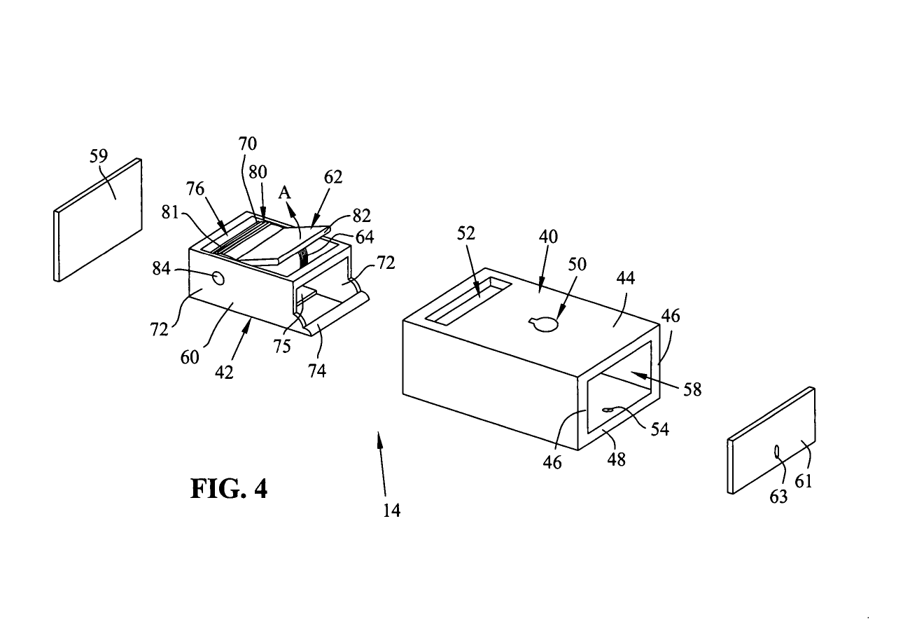

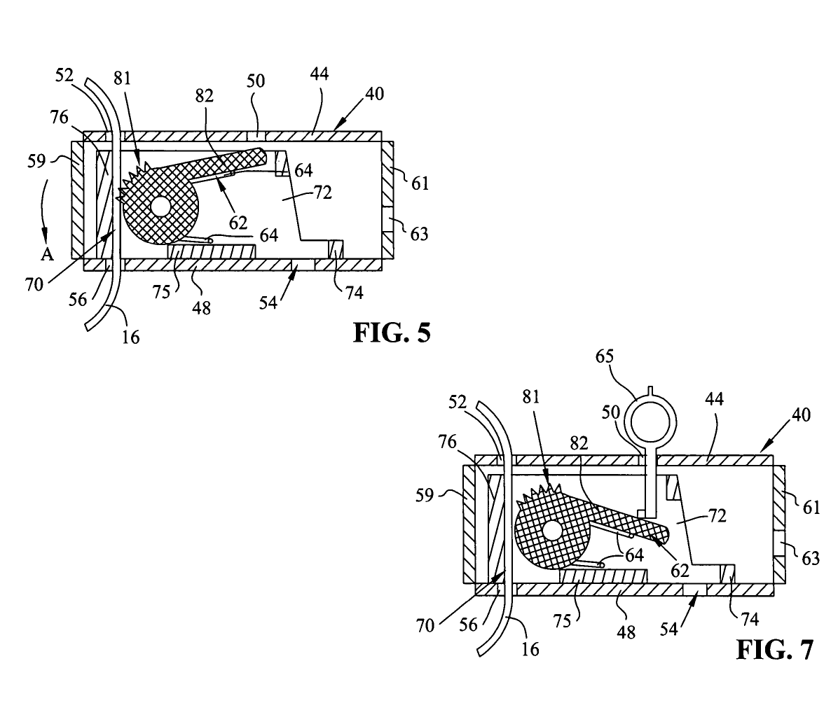

FIGS. 3 and 4 depict a perspective view and an exploded perspective view, respectively, of buckle assembly 14. In the present embodiment, buckle assembly 14 includes a cover 40 and a buckle 42.

Cover 40 is preferably made from metal but may be any material with sufficient strength and durability may be used. Cover 40 includes a first member 44, a pair of walls, each generally indicated by numeral 46, and a second member 48. First member 44 has a rectangular shape and includes a key hole 50 positioned approximately in the center. First member 44 further includes a slot 52 located proximate a short end of the member 44. The side walls 46 extend downward from the long edges of first member 44. Side walls 46 may be attached to first member 44 in any known manner.

Second member 48 has a rectangular shape complementary to first member 44 and is attached to the lower edges of side walls 46 in a known manner. Second member 48 includes an aperture 54 and a slot 56. Slot 56 is positioned in a position directly opposite that of slot 52 in first member 44. Aperture 54 is positioned within second member 48 at an end opposite that of slot 56. The combination of first member 44, side walls 46 and second member 48 define a central receiving area, generally indicated by numeral 58.

In the present embodiment of the invention, cover 40 further includes end portion 59 and end portion 61. The end portions 59, 61 are configured to be attached to the ends of cover 40 in a known manner. In the present embodiment of the invention, end portion 61 includes an aperture 63.

In the present embodiment of the invention, buckle 42 includes a frame 60, a latch 62 and a spring (not shown). Frame 60 includes a strap receiving area, generally indicated by numeral 70, a pair of sides 72, a member 74 and a base 75. Strap receiving area 70 may be of any type known in the art, and in the present embodiment of the invention, strap receiving area 70 includes a rough surface 76. Rough surface 76 has a coarse texture in the present embodiment, but it should be noted that the exact shape and texture of rough surface 76 may be modified, as would be understood by one with ordinary skill in the art.

Sides 72 extend in parallel directions away from strap receiving area 70. Sides 72 each include an aperture (not shown). In the present embodiment of the invention, the apertures in the sides 72 are substantially aligned. Member 74 extends between the sides 72. In the present embodiment of the invention, member 74 joins the lower outer edges of sides 72 together at an end located opposite strap receiving area 70.

Base 75 also interconnects the lower edges of sides 72. Base 75 is located at a position intermediate strap receiving area 70 and member 74.

Latch 62 may have any configuration known in the art, but in the present embodiment of the invention, latch 62 includes a strap engaging portion 80 and a body 82. Strap engaging portion 80 represents a surface having a course texture defined by a plurality of teeth generally indicated by numeral 81. In the present embodiment of the invention, strap engaging portion 80 is positioned at an end of body 82. Furthermore, it should be noted that in the present embodiment of the invention, strap engaging portion 80 may have a shape complementary to that of strap receiving area 70.

Body 82 includes a substantially rectangular portion that extends away from strap engaging portion 80. Body 82 includes a pair of pivot members, each generally indicated by numeral 84. Pivot members 84 extend outward from the outer surfaces of the body 82 and are sized and configured to be received by apertures present within the sides 72 of frame 60. It should be apparent to one with ordinary skill in the art that once the pivot members 84 are received by the apertures, body 82 will be capable of pivoting within frame 60 about the pivot members 84. In the embodiment of the invention depicted, strap engaging portion 80 mates with and engages strap receiving area 70 as body 82 pivots in a first direction, indicated by arrow “A,” if no body or component is positioned intermediate the strap engaging portion 80 and the strap receiving area 70. However, as body 82 pivots about pivot members 84 in a second direction opposite the direction indicated by arrow A, strap engaging portion 80 moves away from the strap receiving area 70.

Latch 62 further includes a spring 64. Spring 64 resides intermediate body 82 and one of the sides 72 of frame 60 and biases body 82 in the direction of arrow A. The pivoting of body 82 toward arrow A ensures that the strap engaging portion 80 is normally biased toward the strap receiving area 70.

In order to assemble device 10, strap 16 is inserted into the strap receiving area 70 of buckle 42. The ends of strap 16 may be pulled through slots 52, 56 from the inside of cover 40. As strap 16 is pulled through slots 52, 56, buckle 42 may be inserted into cover 40. End portion 59, 61 are then attached to cover 40 in order to secure buckle 42 within cover 40. One end of strap 16 is then sewn in a manner forming loop 92. The opposite end of strap 16 is inserted into a ring 34 of a clamp 12. This end of strap 16 is then sewn in a manner forming loop 90 thereby affixing strap 16 to the clamp 12.

The ring 34 of a second clamp 12 is inserted into cover 40 through aperture 63 of end portion 61. Ring 34 exits cover 40 through aperture 54 of bottom member 48. This succeeds in attaching the second clamp 12 to buckle assembly 14 and completes the assembly of the device.

It should be noted that if desired, buckle assembly 14 may be modified in order to fixedly receive a second strap (not shown). The second strap may be connected to the second clamp 12. If desired, the second strap may be adjustably connected to buckle assembly 16 in a manner similar to that described above with respect to first strap 16.

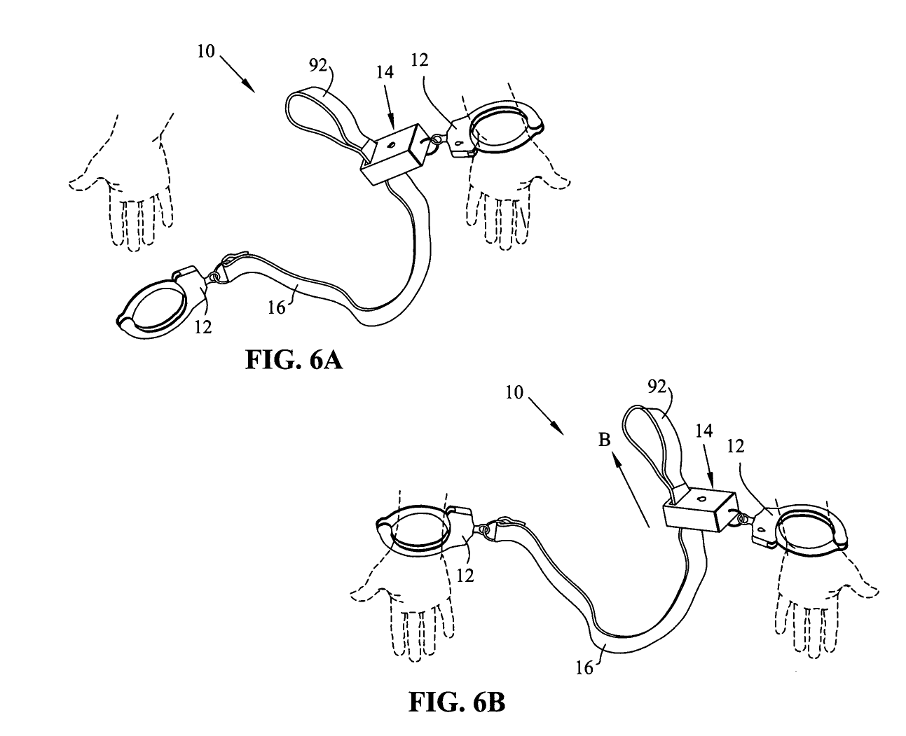

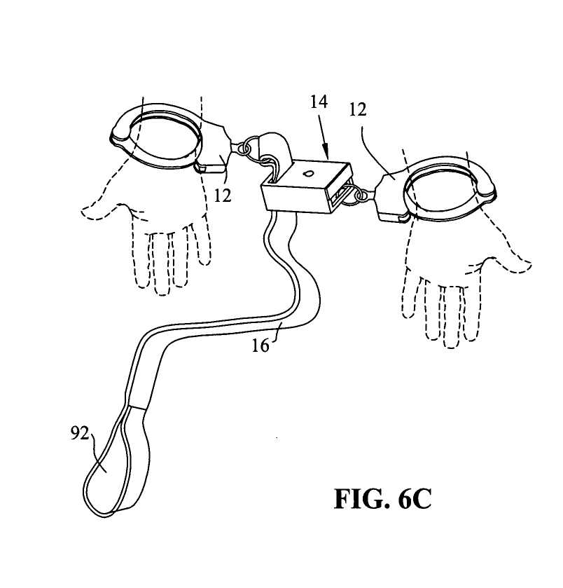

The following description illustrates one method of using device 10 to restrain an individual but should not in any way be construed as limiting the manner in which device 10 may be used. Referring now to FIGS. 6A through 6C, one of the clamps 12 is attached to one wrist of the person to be restrained, as shown in FIG. 6A. This may be done in a known manner. Once clamp 12 has been secured, the other clamp 12 is secured to the other wrist, as shown in FIG. 6B. In addition, it should be noted that in the present demonstration, the strap 16 is initially arranged to ensure that the maximum distance separates the clamps 12. Accordingly, loop 92 is positioned proximate the buckle assembly 14.

Once the clamps 12 have been secured, as shown in FIG. 6B, a law enforcement officer or other person restraining the person may pull loop 92 in the direction of Arrow “A.” As loop 92 is pulled, strap 16 traverses through buckle assembly 14 freely in a first direction. It should be noted, however, that the buckle assembly 14 prevents strap 16 from freely traversing the buckle assembly 14 in a direction opposite the first direction. As the officer continues to pull on loop 92, the clamps 12 are drawn together such that eventually the wrists of the detainee are positioned proximate one another, as depicted in FIG. 6C. In this manner, the detainer is able to restrain the detainee with the arms of the detainee secured behind the back of the detainee and the wrists of the detainee positioned close together. If desired, the wrists may be drawn together in front of the person being restrained. In addition, if desired, the detainer may position the detainee on this stomach and loop 92 may be secured around the foot of the detainee, thereby fully securing the detainee.

Referring still to FIG. 6C, when the detainer desires to release the detainee, the detainer inserts a key (not shown) into the key hole 32 and rotates the key in a known manner. As described above, the rotation of the key releases pivot arm 26 and allows for an increase in the size of the receiving portion 22. Once the detainer has sufficiently increased the size of the receiving portion 22, the detainer may remove the adjustable restraints 10 from the detainee, thereby releasing the detainee.

In the alternative, if the detainer desires to provide additional freedom of movement to the detainee but does not desire to free the detainee, the detainer may insert a key 65 into the key hole 50 of cover 40, as shown in FIG. 7. Upon insertion, the key contacts the distal end of latch 62. The force the key imparts on latch 62 causes latch 62 to pivot about pivot members 84 against the biasing of spring 64. The pivoting of the body 82 about the pivot members 84 moves strap engaging portion 80 away from strap receiving area 70. This movement thereby allows strap 16 to travel freely through buckle assembly 14. The travel of strap 16 through buckle assembly 14 allows the detainer to increase the distance separating the clamps 12. In this manner, the detainer may provide the detainee additional limited freedom of movement by increasing the separation distance of the wrists of the detainee.

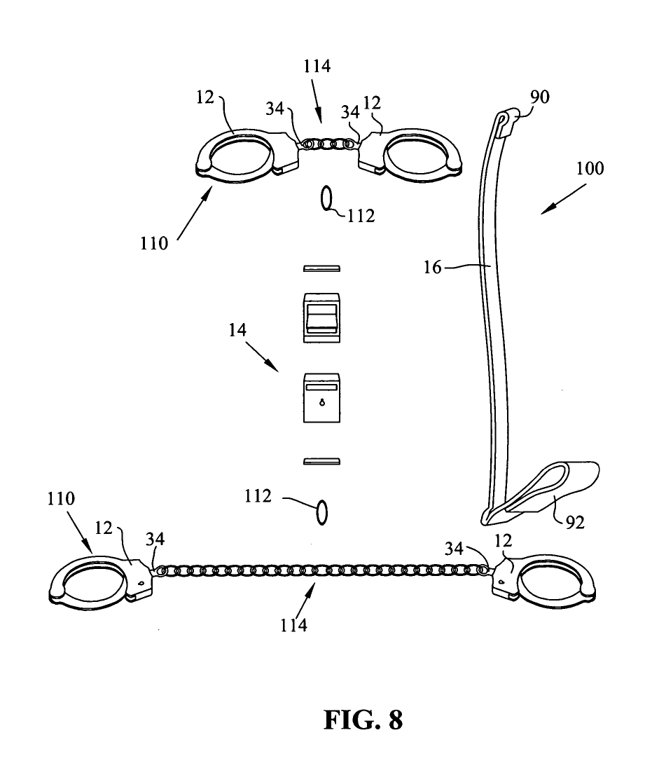

FIG. 8 depicts an alternative embodiment of the present invention, generally indicated by numeral 100. In an effort to simplify the present description, only those components differing from those described previously, will be described with respect to device 100. In the present embodiment, device 100 includes a buckle assembly 14, a strap 16, a pair of assemblies, each generally indicated by numeral 110 and a pair of rings, each generally indicated by numeral 112.

In the present embodiment, assemblies 110 include a pair of clamps 12 interconnected by a chain 114. Chain 114 may be any type having sufficient strength and may be attached to the rings 34 of clamps 12 in any suitable manner. In addition, the length of chain 114 may vary as desired, from a size of a single link to multiple links.

One of the rings 112 attaches strap 16 to chain 114 of a first of the assemblies 110, in a manner similar to that described above in the previous embodiment. Specifically, loop 90 of strap 16 connects strap 16 to ring 112. Referring still to FIG. 8, a ring 112 connects the other of assemblies 110 to buckle assembly 14, in a manner similar to that described above with respect to the previous embodiment.

Device 100 may be assembled in a manner similar to that described above with respect to the previous embodiment of the invention. In the present embodiment, however, the rings 112 connect the chain 114 to the buckle assembly 14 and strap 16, in a manner similar to the connection of the clamps 12 in the previous embodiment. The remainder of the assembly process remains substantially identical to the previous embodiment.

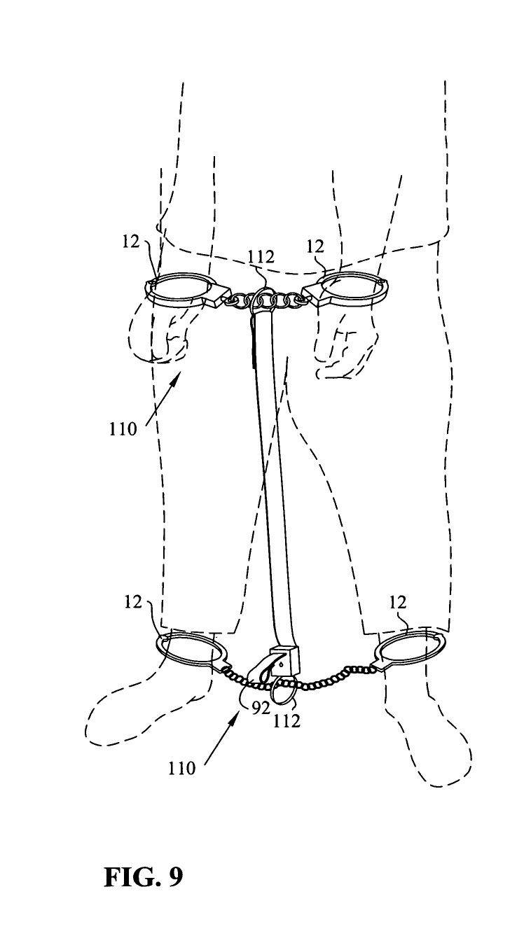

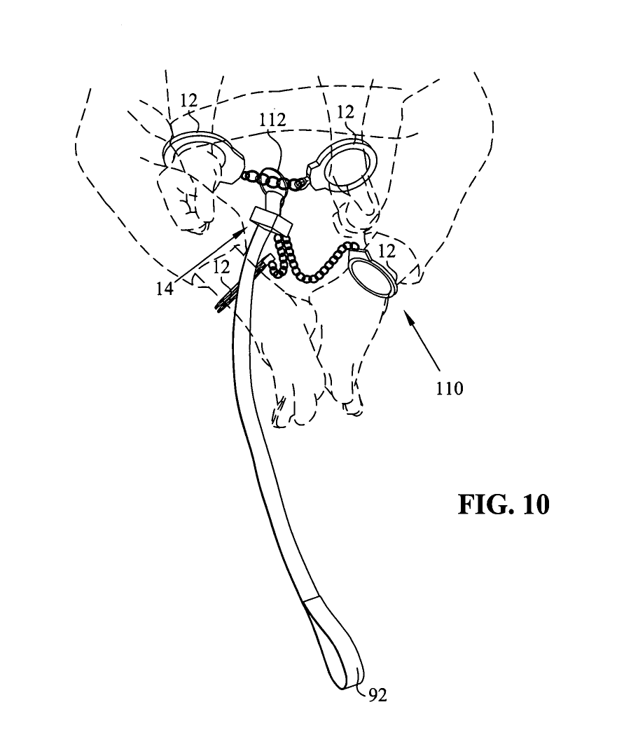

FIG. 9 depicts device 100 in use. The clamps 12 of one assembly 110 are attached to the wrists of the detainee. The clamps 12 of the other assembly 110 are attached to the ankles of the detainee. Once the device 100 has been affixed to a detainee, the detainee is able to shuffle his feet thereby allowing for travel at a limited speed. In addition, the assembly 110 attached to the wrists of the detainee permits limited movement of the arms of the detainee. If the detainee chooses to act in an inappropriate manner, a detainer may pull on loop 92 of strap 16, thereby forcing the detainee into the seated and restricted position depicted in FIG. 10, with the wrists of the detainee retained in a position proximate the ankles of the detainee. Once placed in this position, the movement of the detainee largely is restricted.

The detainer may release the detainee in a manner similar to that described above with respect to the previous embodiment. Specifically, a key may be used to release the wrists and ankles of the detainee. In addition, if the detainer desires to provide additional freedom to the detainee without providing unlimited freedom, the detainer may increase the distance separating the wrists and ankles of the detainee by utilizing a key (not shown) to actuate the buckle assembly 14, in a manner similar to that described above with respect to the previous embodiment of the invention.

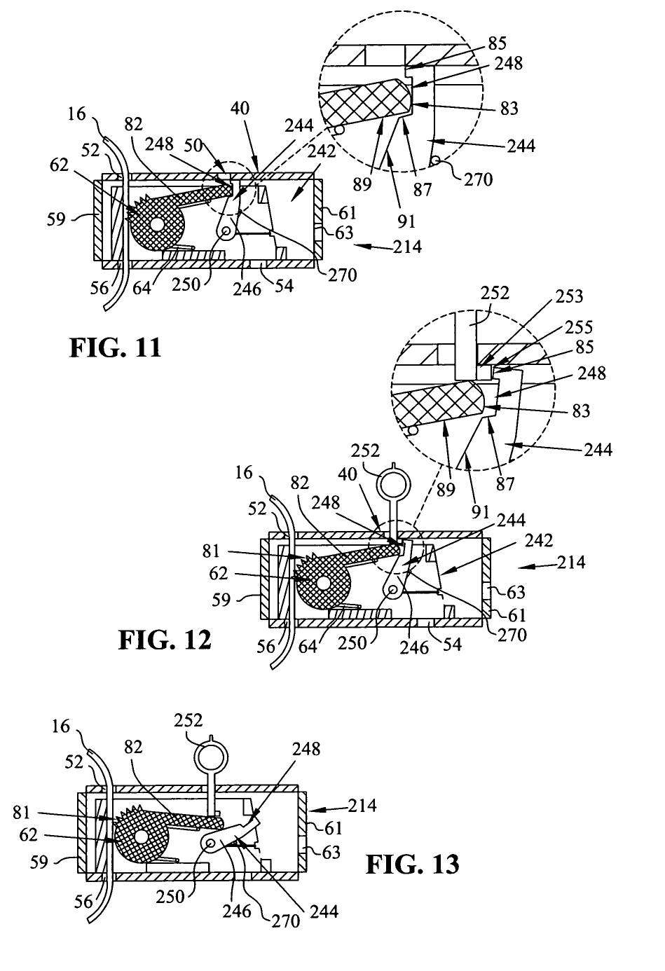

FIG. 11 depicts a cross section of an alternative embodiment of a buckle assembly, generally indicated by numeral 214. For the sake of clarity, only components differing from those in the previous embodiment of buckle assembly 14 will be described. Buckle assembly 214 includes a cover 40 and a buckle 242.

Buckle 242 has a configuration substantially similar to buckle 42. In the present embodiment, buckle 242 includes a pawl 244. Pawl 244 includes a body portion 246 with a notch 248 and a pin 250. Pin 250 extend through apertures (not shown) present within side walls 72 of buckle 242.

As can be seen in FIG. 11, pawl 244 is sized and configured to receive an end 83 of body 82 within notch 248. As would be understood by one with skill in the art, when pawl 244 retains end 83, pawl 244 prevents latch 62 from rotating.

When one desires to rotate latch 62 in order to move buckle assembly 214 as described above, one inserts key 252 into key hole 50 until key 252 contacts the upper surface of body 82. Key 252 may then be rotated into engagement with pawl 244 in a manner causing key portion 253 to contact upper surface 85 of pawl 244. The contact between key portion 253 and upper surface 85 causes rotation of pawl 244 to a position wherein end 83 becomes clear of surface 87 of notch 248, thereby freeing end 83 from pawl 244 as shown in FIG. 12.

Once end 83 of body 82 is free from pawl 244, one may insert key 252 further into cover 40 in order to create rotation of body 82. As body 82 rotates, end 83 of body 82 traverses front edge 91 of pawl 244. This movement causes pawl 244 to continue rotating until the position of the pawl 244 and body 82 is that depicted in FIG. 13. The rotation of body 82, as depicted, causes teeth 81 to disengage strap 16, thereby allowing buckle assembly 214 to moved along strap 16 in any direction. Once buckle assembly 214 has been located in the desired position, one may withdraw key 252 and a spring 270 will cause pawl 244 to rotate into the position depicted in FIG. 11. End 83 will traverse surface 91 and engage again notch 248, thereby preventing movement of latch 62 as described above. The inclusion of pawl 244 prevents one from releasing latch 62 with any small elongated object.

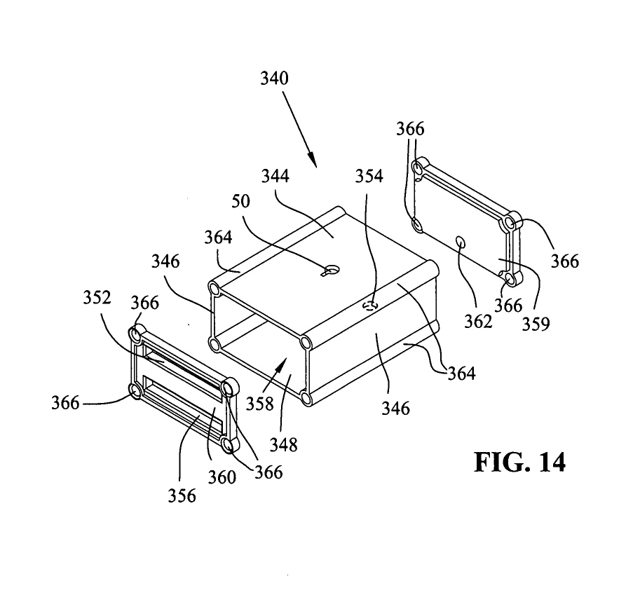

FIG. 14 depicts an alternative embodiment of a cover, generally indicated by numeral 340. Cover 340 includes a first member 344, a pair of side walls 346, a second member 348, end portion 359 and end portion 360. In the present embodiment of the invention, the side walls 346 are substantially similar to that described previously but include fastener receiving channels 364.

First member 344 has a rectangular shape and includes a key hole 50 but does not include a slot as in previous embodiments. Similarly, second member 348 has a complementary rectangular shape and includes an aperture 354, as in the previous embodiment. In the present embodiment, second member 348 does not include a slot. First member 344, walls 346 and second member 348 may be joined as described above with respect to the previous embodiment of the invention, in order to define buckle receiving area 358. The components comprising cover 340 may all be manufactured as individual components, or may be completed integrated and cover 340 may be a one piece extrusion.

In the present embodiment, end portion 359 and end portion 360 are sized to cover the openings of area 358. End portion 360 includes an aperture 362, and end portion 359 and end portion 360 each include four fastener receiving apertures 366.

In the present embodiment, end portion 360 has a shape substantially identical to end portion 359. End portion 359 includes an upper slot 352 and a lower slot 356. The slots 352, 356 are sized to allow strap 16 to extend therethrough. Cover 340 may be utilized with embodiments of the present invention wherein it is desirous for strap 16 to enter into and extend out from one end of cover 340. Fasteners or other means known to someone skilled in the art can attach the end portions 359, 360 by extending through the apertures 366 and into channels 364.

The inventors anticipate that a Global Positioning System, or similar type locating system, may be incorporated into the covers of the buckle assembly. For example, the cover may include a chip that, upon activation, sends out a signal allowing a police officer to utilize a scanner in order to locate quickly a person wearing an embodiment of the present invention. In addition, the cover may also include components capable of producing a loud audible sound upon receiving a signal, in order to allow officers to locate a person who has escaped from police control while wearing an embodiment of the device.

While this invention has been described as having an exemplary design, the present invention may be further modified within the spirit and scope of this disclosure. The application is intended, therefore, to cover any variations, uses, or adaptations of the invention using its general principles. Further, this application is intended to cover such departures from the present disclosure as come within known or customary practice in the art to which this invention pertains.

We claim:

1. A restraining device comprising:
a first clamp connected to a strap; and
a second clamp connected to a buckle assembly configured to receive the strap;
wherein the buckle assembly can move along the strap in a first direction.

2. The restraining device as set forth in claim 1 wherein the buckle assembly can move along the strap in a second direction opposite the first direction when actuated.

3. The restraining device as set forth in claim 2 wherein the buckle assembly is actuated by a key.

4. The restraining device as set forth in claim 3 wherein the size of the clamps are adjustable in size when actuated by the key.

5. The restraining device as set forth in claim 1 wherein the buckle assembly includes a latch and a cover.

6. The restraining device as set forth in claim 5 wherein the cover includes a pair of slots.

7. The restraining device as set forth in claim 6 wherein one of the slots is formed in a surface of the buckle assembly including a keyhole and the other slot is formed in a surface of the buckle assembly opposite the surface including the keyhole.

8. The restraining device as set forth in claim 6 wherein the pair of slots are formed in a single surface.

9. The restraining device as set forth in claim 1 further including a third and a fourth clamp

10. The restraining device as set forth in claim 9 further including a first chain connecting the first clamp to the third clamp and a second chain connecting the second clamp to the fourth clamp.

11. The restraining device as set forth in claim 10 further including a ring connecting one of the chains to the strap.

12. The restraining device as set forth in claim 11 further including a second ring connecting the other chain to the buckle assembly.

13. The restraining device as set forth in claim 1 wherein the strap includes a loop formed opposite the first clamp.

14. A restraining device including:
a first clamp connected to a strap including a loop; and
a second clamp connected to a buckle assembly;
wherein a first set distance separating the first and second clamps decreases when the loop is pulled.

15. The restraining device as set forth in claim 14 wherein the first set distance may be increased when the buckle assembly is actuated.

16. The restraining device as set forth in claim 15 including a key for releasing the buckle assembly.

17. The restraining device as set forth in claim 14 wherein the loop is formed in the strap opposite the first clamp.

18. The restraining device as set forth in claim 14 wherein the buckle assembly includes at least one slot and the strap extends through the at least one slot.

19. The restraining device as set forth in claim 18 wherein the buckle assembly includes a pair of slots.

20. The restraining device as set forth in claim 14 wherein the buckle assembly includes a latch and a locking pawl, the latch preventing movement of the strap through the buckle assembly and the locking pawl preventing movement of the latch.

21. The restraining device as set forth in claim 20 wherein the buckle assembly further includes a strap receiving area, the strap being located between the strap receiving area and the latch.

22. A method of restraining an individual comprising the steps of:
affixing a first clamp to a first wrist of the individual;
affixing a second clamp to a second wrist of the individual, the second clamp separated from the first clamp by a first set distance; and
pulling a loop formed in a strap interconnecting the first clamp to the second clamp in order to decrease the maximum allowable distance.

23. The method of restraining an individual as set forth in claim 22 further comprising the step of affixing the loop to a foot of the individual.

24. The method of restraining an individual as set forth in claim 22 further comprising the step of actuating a buckle assembly in order to allow for an increase in the first set distance.

25. A device for restraining an individual including:
a strap having a pair of loops formed at its ends;
a buckle assembly capable of moving along the strap;
at least one clamp attached to the buckle assembly; and
at least one clamp attached to one of the loops formed in the strap.
